(19) United States
(12) Patent Application Publication
(10) Pub. No.: US 2023/0147012 A1
(43) Pub. Date: May 11, 2023

(54) SYSTEMS AND METHODS FOR DETERMINING ALTERNATIVE PAYMENTS FOR PHARMACEUTICAL THERAPIES AND DRUGS

(71) Applicant: Quantile Health, Inc., New York, NY (US)

(72) Inventor: Yutong Sun, New York, NY (US)

(73) Assignee: Quantile Health, Inc., New York, NY (US)

(21) Appl. No.: 17/541,067

(22) Filed: Dec. 2, 2021

Related U.S. Application Data
(60) Provisional application No. 63/263,650, filed on Nov. 5, 2021.

(51) Int. Cl. G16H 50/20; G16H 20/10; G16H 10/60; G06N 20/00; G06Q 20/22; G06Q 20/10

(52) U.S. Cl. CPC G16H 50/20; G16H 20/10; G16H 10/60; G06N 20/00; G06Q 20/22; G06Q 20/102

(57) ABSTRACT

Systems and methods for determining alternative payments for pharmaceutical therapies and drugs are disclosed herein. An example system for determining a payment includes receiving a first set of input data associated with a disease, determining a plurality of disease-specific models associated with the disease based at least in part on the input data, determining a baseline disease model associated with the disease based on the plurality of disease-specific models, receiving a second set of input data, determining a user adjustment model associated with a customer based at least in part on the second set of input data, and calculating a subscription prescription payment for the customer based at least in part on the baseline disease model, the second set of input data, and the user adjustment model.

### CROSS-REFERENCE TO RELATED APPLICATION(S)

This application claims the benefit of U.S. Provisional Application No. 63/263,650, filed Nov. 5, 2021, the disclosure of which is incorporated herein by reference.

### TECHNICAL FIELD

The present disclosure is generally directed to alternative payments for pharmaceutical drugs and therapies, and, more particularly, to systems and methods for determining those alternative payments.

### BACKGROUND

Most pharmaceutical therapies in the United States are paid for under a traditional “fee-for-service” model. However, payers, pharmaceutical companies, and regulatory agencies are increasingly looking to shift away from “fee-for-service” models and towards alternative payment methods, including, but not limited to, subscription payment models, warranty models, and variants of the “fee-for-service” models where quality metrics may trigger additional payments or rebates. Such a shift is partially motivated by the emergence of a new class of highly expensive and innovative gene therapies, which have uniquely wide distributions of potential costs. During the underwriting process, payers often model utilization for a therapy as a random variable (e.g., a number of drugs or therapies needed in a payer population), and that utilization variable is then scaled by a constant (e.g., a fee-for-service price of a pharmaceutical drug or therapy) to project another random variable (e.g., estimated total costs of the pharmaceutical drug or therapy). These random variables can have a much higher variance than most medical treatments, so payers often purchase re-insurance in order to protect themselves from a tail risk outcome. However, the high variance has resulted in re-insurers charging payers a large multiple of the expected medical cost, and this cost is thus passed onto consumers. Thus, the total cost of adding these innovative drugs or therapies to a policy may be higher than an expected cost of the drugs or therapies themselves. Because of the prohibitively high cost of re-insurance, payers may be dissuaded from covering these potentially curative pharmaceutical drugs or therapies, which limits consumer access to potentially life-saving treatments.

Payment methods, such as subscription payments, can dramatically reduce the distribution of potential payments, thus lowering costs for payers, which reduces costs and increases access associated with patients of such pharmaceutical therapies or drugs. However, in order for these alternative payment methods to become widely adopted, the alternative payments need to be supported by certain systems and methods of estimating utilization and associated costs and of tracking patient outcomes. Thus, a model for determining alternative payments, along with systems and methods for estimating utilization and associated costs, constitutes a substantial technical improvement over existing systems and methods in order to lower consumer costs, accurately track patient outcomes, and generate utilization predictions.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 illustrates a flow diagram of an example process for calculating a clinical outcome score in accordance with one example embodiment of the disclosure.

FIG. 2 illustrates flow diagrams of example processes for determining a baseline pricing model and calculating a subscription prescription payment in accordance with one example embodiment of the disclosure.

FIG. 3 illustrates a flow diagram of an exemplary methodology for calculating a clinical outcome score in accordance with one example embodiment of the disclosure.

FIG. 4 illustrates a flow diagram of an exemplary methodology for calculating a subscription prescription payment in accordance with one example embodiment of the disclosure.

FIG. 5 illustrates an example computing device in accordance with one example embodiment of this disclosure.

This disclosure will now provide a more detailed and specific description that will refer to the accompanying drawings. The drawings and specific descriptions of the drawings, as well as any specific or other embodiments discussed, are intended to be read in conjunction with the entirety of this disclosure.

### DETAILED DESCRIPTION OF THE EXAMPLE EMBODIMENTS

Example embodiments will now be described more fully hereinafter with reference to the accompanying drawings, in which example embodiments are shown. The concepts disclosed herein may, however, be embodied in many different forms and should not be construed as limited to the example embodiments set forth herein; rather, these embodiments are provided so that this disclosure will be thorough and complete, and will fully convey the scope of the concepts to those skilled in the art. Like numbers may refer to like, but not necessarily the same or identical elements throughout.

The terms “pharmaceutical” and “pharmaceutical drug” can be used interchangeably herein, and refer to any substance or compound that may be used for any medicinal purpose, treatment, or therapy.

This disclosure relates to, among other things, systems and methods for determining alternative payments for pharmaceutical therapies and drugs. In certain embodiments, systems and methods can determine a pricing model that is configured to determine a subscription prescription payment associated with a pharmaceutical or other therapy, and to further determine a clinical outcome score associated with a pharmaceutical or other therapy. In other embodiments, systems and methods can determine a fee-for-service model in which fee-for-service payments can be modified based on clinical markers in an individual or population. In other embodiments, systems and methods can determine a warranty model in which a pharmaceutical drug or therapy manufacturer can provide a warranty that certain clinical markers will be met (e.g., survival, a maximum number of days stayed in the hospital, a maximum number of readmissions, the costs of other therapies having a maximum price under a predetermined threshold, etc.), where payers can be compensated for services required to achieve warranty status. Such systems and methods can be applied to diseases including, but not limited to, hemophilia A, hemophilia B, sickle cell, spinal muscular atrophy, cerebral adrenolrukodystrophy, leber's hereditary optic neuropathy, TDBT beta-thalassemia major, and leukemia. In some embodiments, each model may be executed on an annual basis, or any other basis associated with a predefined time period, in order to increase the accuracy of each model.

Embodiments of the disclosure can provide a technical solution to the problem of accurately calculating a pricing of a subscription prescription payment and a clinical outcome score for pharmaceuticals or other therapies.

In one example embodiment, a method for calculating a clinical outcome score can be provided.

In one embodiment, a first set of input data, which may include clinical trial data, scientific research data, patient advocacy data, and/or external testing data, can be used to determine a therapy outcome measurement at year zero associated with a therapy or a pharmaceutical. As an example, in a case for treatment for infants with SMA Type I, the therapy outcome measurement may reflect the percentage of patients across all observed populations who are alive and on a ventilator one year after the onset of treatment.

In one embodiment, a second set of input data associated with a consumer or a potential consumer can be received. In one embodiment, the medical data can include historical medical claims data and/or medical charts of potential consumers.

In one embodiment, a clinical outcome measurement model can be determined. In one embodiment, the clinical outcome measurement model can involve a machine learning algorithm.

In one embodiment, medical review may be conducted by physicians and/or professionals in a relatively rare disease field. As an example, in a case for treatment for patients with SMA Type I, medical review may include individual assessments of each patient's motor skills by physicians unaffiliated with any interested parties (e.g., pharmaceutical drug or therapy manufacturers, payers, or re-insurers).

In one embodiment, a clinical outcome score for the therapy or the pharmaceutical can be calculated based on the clinical trial data, the scientific research data, the patient advocacy data, the external testing, the therapy outcome measurement at year zero, the medical data, the clinical outcome measurement model, and/or the medical review.

In one embodiment, if a machine learning algorithm is used to calculate the clinical outcome score, a first weight can be associated with the first set of input data, a second weight can be associated with the therapy outcome measurement at year zero, a third weight can be associated with the second set of input data, and a fourth weight can be associated with the clinical outcome measurement model. The clinical outcome score can then be calculated based at least in part on the first set of input data, the therapy outcome measurement at year zero, the second set of input data, the clinical outcome measurement model, the medical review, the first weight, the second weight, the third weight, and/or the fourth weight.

In one embodiment, the clinical outcome score can be displayed at a graphical user interface (GUI).

In one example embodiment, a system for calculating a subscription prescription payment for a therapy or a pharmaceutical can be provided.

In one embodiment, a first set of input data can be received. The first set of input data can include disease-specific demographic data and/or metadata associated with the disease.

In one embodiment, the disease-specific demographic data can include claim data associated with the therapy or the pharmaceutical, prescription data, patient advocacy data, and/or healthcare provider data. As an example, disease-specific demographic data associated with SMA Type I may include the patient's age and state of residence at the time of diagnosis for each patient in the United States.

In one embodiment, metadata associated with a disease can be received. In one embodiment, the metadata can include patient prevalence research, physician input, and/or generic screening and testing. As an example, metadata associated with patient prevalence of sickle cell may be based at least in part on academic publications and conversations with experts in the field about screening methodologies for detecting sickle cell, and may further account for the uneven quality in screening throughout the United States.

In one embodiment, a plurality of disease-specific models associated with a disease can be determined based at least in part on the first set of input data. In one embodiment, the at least one model can include a symptom progression model, a diagnostic pattern model, a treatment model, and/or a care management model. In one embodiment, the symptom progression model can create a distribution that reflects the possible evolution of symptoms for a patient and a population over time for a given disease. For example, with respect to patients having SMA Type I, the symptom progression model may reflect a change in patients' motor ability over time if the disease is left untreated, and it may include probabilities that a patient may require a ventilator after receiving a given treatment. In one embodiment, the diagnostic pattern model can estimate the proportion of individuals in a population that are diagnosed with a given disease. For example, with respect to specific types of leukemia, a diagnostic pattern model may estimate the proportion of patients that have been diagnosed with said specific types of leukemia. In one embodiment, a treatment model can estimate a distribution of potential treatment paths for a given disease and symptom progression. For example, with respect to a specific type of leukemia, a treatment model may reflect different probabilities associated with different therapies at different time intervals following diagnosis of the patient.

In one embodiment, a baseline disease model can be determined based at least in part on the plurality of disease-specific models.

In one embodiment, a second set of input data can be received. The second set of input can include data associated with a payer population and/or customer-specific policy data.

In one embodiment, the data associated with a payer population can include payer demographic data and/or historical claim data associated with the payer population. As an example, payer demographic data can include the age, sex, and/or known medical conditions of all members of the payer population.

In one embodiment, customer-specific policy data can include payer-specific coverage policy data associated with the customer and/or payer-specific reinsurance policy data associated with the customer. As an example, a payer may have a coverage policy that restricts the use of a novel therapy to a more limited set of clinical situations than another person. Such situations may result in a shift in the distribution of expected utilization and expected cost of a drug or therapy

In one embodiment, a user adjustment model can be determined based at least in part on the second set of input data.

In one embodiment, a subscription prescription payment for the therapy or the pharmaceutical drug can be calculated based on the baseline disease model, the second set of input data, and/or the user adjustment model. In one embodiment, this payment can be a per-member-per-month fee.

In one embodiment, the subscription prescription payment can be calculated by using a machine learning algorithm. The first set of input data can be associated with a first weight, the plurality of disease-specific models can be associated with a second weight, the baseline disease model can be associated with a third weight, the second set of input data is associated with a fourth weight, and the user adjustment model can be associated with a fifth weight. The subscription prescription payment can be calculated based at least in part on the first set of input data, the plurality of disease-specific models, the baseline disease model, the second set of input data, the user adjustment model, the first weight, the second weight, the third weight, the fourth weight, and the fifth weight.

In one embodiment, the subscription prescription payment can be displayed at a graphical user interface (GUI).

In one embodiment, an exemplary methodology for calculating a subscription prescription payment for a therapy or a pharmaceutical drug can be provided.

In one embodiment, a first set of input data associated with a therapy or a pharmaceutical can be received. In one embodiment, the first set of input data can include disease-specific demographic data and metadata. The disease-specific demographic data can include claim data associated with a disease, prescription data associated with the disease, patient advocacy data associated with the disease, and/or healthcare provider data associated with the disease. The metadata can include patient prevalence research associated with the disease, physician input associated with the disease, and/or generic screening and testing associated with the disease.

In one embodiment, a first weight can be assigned to the first set of input data.

In one embodiment, a plurality of disease-specific models associated with the disease can be determined based at least in part on the first set of input data. The plurality of disease-specific models can include a symptom progression model associated with the disease, a diagnostic pattern model associated with the disease, a treatment model associated with the disease, and/or a care management model associated with the disease

In one embodiment, a second weight can be assigned to the plurality of disease-specific models.

In one embodiment, a baseline disease model associated with the disease can be determined based on the plurality of disease-specific models.

In one embodiment, a third weight can be assigned to the baseline disease model.

In one embodiment, a second set of input data can be received. The second set of input data can include data associated with a payer population and customer-specific policy data. In one embodiment, the data associated with a payer-population can include payer demographic data associated with the payer population and/or historical claim data associated with the payer population. In one embodiment, the customer-specific policy data can include payer-specific coverage policy data associated with the customer and/or payer-specific reinsurance policy data associated with the customer.

In one embodiment, a fourth weight can be assigned to the second set of input data.

In one embodiment, a user adjustment model associated with a customer can be determined based at least in part on the second set of input data.

In one embodiment, a fifth weight can be assigned to the user adjustment model.

In one embodiment, a subscription prescription payment for the customer can be calculated based at least in part on the baseline disease model, the second set of input data, the user adjustment model, the first weight, the second weight, the third weight, the fourth weight, and the fifth weight.

In one embodiment, the subscription prescription payment for the therapy or pharmaceutical can be displayed at a graphical user interface (GUI).

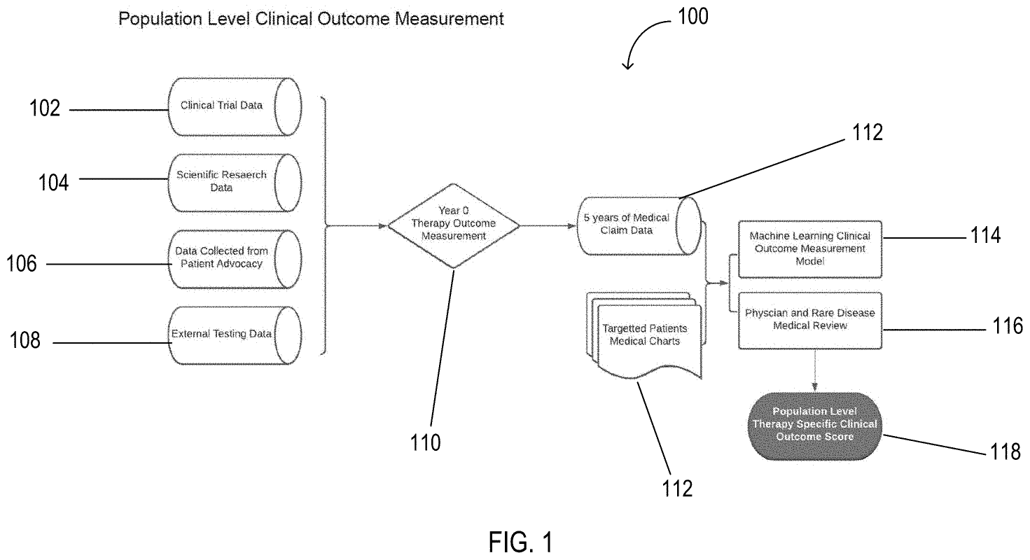

Turning to the figures, FIG. 1 is a flow diagram of an example system 100 for calculating a clinical outcome score, according to an embodiment of the disclosure. As depicted in FIG. 1, in some embodiments, clinical trial data 102, scientific research data 104, patient advocacy data 106, and/or external testing data 108 associated with a therapy or a pharmaceutical can be collected and used to determine a therapy outcome measurement at year zero 110. In some embodiments, the therapy outcome measurement at year zero 110 is determined based on clinical trial data 102, scientific research data 104, patient advocacy data 106, and external testing data 108. In other embodiments, the therapy outcome measurement at year zero 110 is determined based on at least one of clinical trial data 102, scientific research data 104, patient advocacy data 106, and external testing data 108. In other embodiments, the therapy outcome measurement at year zero 110 is determined based on a combination of clinical trial data 102, scientific research data 104, patient advocacy data, and/or external testing data 108. In other embodiments, the therapy outcome measurement at year zero 110 is determined based on a combination of clinical trial data 102, scientific research data 104, patient advocacy data, external testing data 108, and/or other types of data. In some embodiments, in a case for treatment for infants with SMA Type I, the therapy outcome measurement at year zero 110 may reflect the percentage of patients across all observed populations who are alive and on a ventilator one year after the onset of treatment.

In some embodiments, medical data 112 associated with a consumer or a potential consumer can received. In some embodiments, the medical data 112 can include historical medical claims data and/or medical charts of potential consumers. In some embodiments, the historical medical claims data can be received for a predetermined amount of time.

In some embodiments, a clinical outcome measurement model 114 can be determined. In certain embodiments, a machine learning algorithm may be implemented in the clinical outcome measurement model 114.

In certain embodiments, medical review 116 may be optionally conducted by physicians and/or professionals in a relatively rare disease field. In some embodiments, in a case for treatment for patients with SMA Type I, medical review 116 may include individual assessments of each patient's motor skills by physicians unaffiliated with any interested parties (e.g., pharmaceutical drug or therapy manufacturers, payers, or re-insurers).

In some embodiments, a clinical outcome score 118 for the therapy or pharmaceutical can be calculated based on the clinical trial data 102, the scientific research data 104, the patient advocacy data 106, the external testing data 108, the therapy outcome measurement at year zero 110, the medical data 112, the clinical outcome measurement model 114, and the medical review 116. In some embodiments, only a subset of the clinical trial data 102, the scientific research data 104, the patient advocacy data 106, the external testing data 108, the therapy outcome measurement at year zero 110, the medical data 112, the clinical outcome measurement model 114, and the medical review 116 can be used to calculate the clinical outcome score 118. In other embodiments, additional considerations can be used to calculate the clinical outcome score 118.

Although not depicted in FIG. 1, in some embodiments, the clinical outcome score 118 can be configured to be displayed at a graphical user interface (GUI).

Although not depicted in FIG. 1, in some embodiments, the clinical outcome score 118 can be updated periodically based on changes to the clinical trial data 102, the scientific research data 104, the patient advocacy data 106, the external testing data 108, the therapy outcome measurement at year zero 110, the medical data 112, the clinical outcome measurement model 114, and the medical review 116.

Although not depicted in FIG. 1, in some embodiments, the clinical trial data 102, the scientific research data 104, the patient advocacy data 106, the external testing data 108, the therapy outcome measurement at year zero 110, the medical data 112, the clinical outcome measurement model 114, and the medical review 116 may be weighted differently when calculating the clinical outcome score 118. A user may manually define weights, or weights may be automatically defined using various machine learning algorithms.

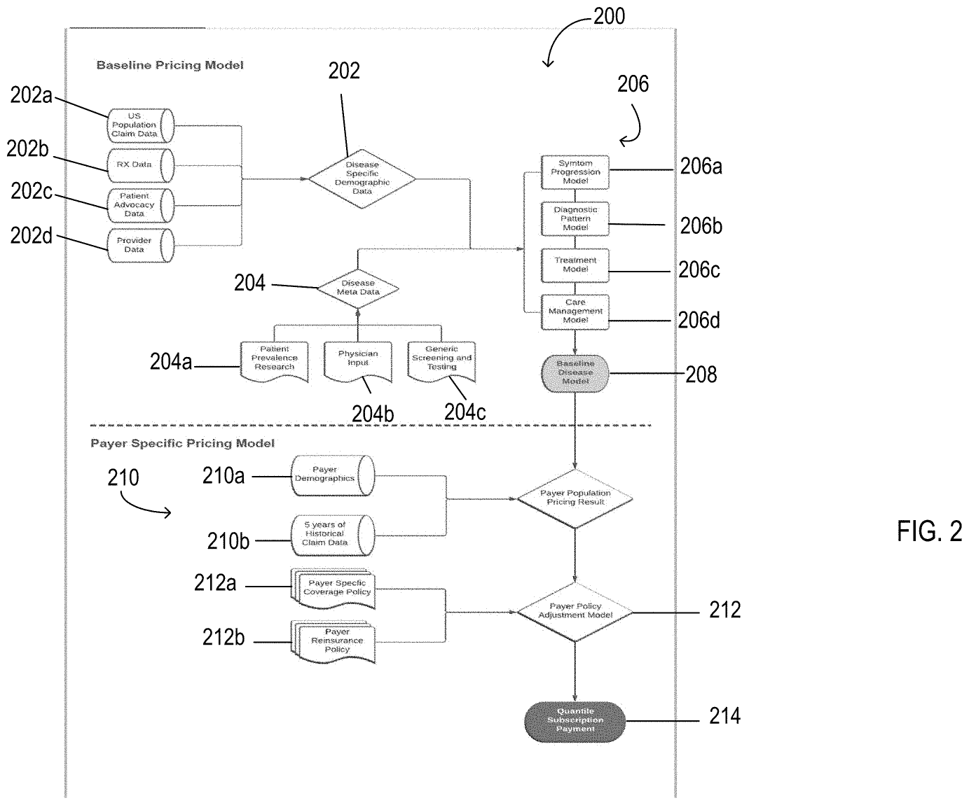

FIG. 2 is a flow diagram of an example process 200 for calculating a subscription prescription payment associated with a therapy or a pharmaceutical, according to an embodiment of the disclosure. As depicted in FIG. 2, in some embodiments, disease-specific demographic data 202 associated with a disease can be received. In some embodiments, disease-specific demographic data 202 can include claim data associated with the therapy or the pharmaceutical 202a, prescription data 202b, patient advocacy data 202c, and/or healthcare provider data 202d. In some embodiments, only a subset of the claim data associated with the therapy or the pharmaceutical 202a, prescription data 202b, patient advocacy data 202c, and/or healthcare provider data 202d can be received. In other embodiments, other types of disease-specific demographic data 202 can be received. In some embodiments, disease-specific demographic data 202 associated with SMA Type I may include the patient's age and state of residence at the time of diagnosis for each patient in the United States. In some embodiments, patient advocacy data 202c may include an additional estimate of patient prevalence in the population.

In some embodiments, metadata 204 associated with a disease can be received. In some embodiments, metadata 204 can include patient prevalence research 204a associated with the disease, physician input 204b, and generic screening and testing 204c associated with the disease. In some embodiments, only a subset of the patient prevalence research 204a, physician input 204b, and/or generic screening and testing 204c can be received. In other embodiments, other types of metadata 204 can be received. In some embodiments, metadata 204 associated with patient prevalence of sickle cell may be based at least in part on academic publications and conversations with experts in the field about screening methodologies for detecting sickle cell, and may further account for the uneven quality in screening throughout the United States.

In some embodiments, at least one model 206 associated with a disease can be determined. In some embodiments, the at least one model 206 can include a symptom progression model 206a, a diagnostic pattern model 206b, a treatment model 206c, and/or a care management model 206d. In some embodiments, only a subset of the a symptom progression model 206a, a diagnostic pattern model 206b, a treatment model 206c, and/or a care management model 206d can be determined. In other embodiments, other models 206 associated with a disease can be determined.

In some embodiments, the symptom progression model 206a can create a distribution that reflects the possible evolution of symptoms for a patient and a population over time for a given disease. For example, with respect to patients having SMA Type I, the symptom progression model 206a may reflect a change in patients' motor ability over time if the disease is left untreated, and it may include probabilities that a patient may require a ventilator after receiving a given treatment. In some embodiments, the diagnostic pattern model 206b can estimate the proportion of individuals in a population that are diagnosed with a given disease. For example, with respect to specific types of leukemia, a diagnostic pattern model 206b may estimate the proportion of patients that have been diagnosed with said specific types of leukemia. In some embodiments, a treatment model 206c can estimate a distribution of potential treatment paths for a given disease and symptom progression. For example, with respect to a specific type of leukemia, a treatment model 206c may reflect different probabilities associated with different therapies at different time intervals following diagnosis of the patient.

In some embodiments, a baseline disease model 208 can be determined based at least in part on disease-specific demographic data 202, the metadata 204, and the at least one model 206. In some embodiments, an output of the baseline disease model 208 may include a distribution of a number of people in a generic population with a disease or a distribution of potential costs in a generic population.

In some embodiments, data associated with a payer population 210 can be received. In some embodiments, the data associated with the payer population 210 can include payer demographic data 210a and historical claim data associated with the payer population 210b. In some embodiments, only a subset of the payer demographic data 210a and historical claim data associated with the payer population 210b can be received. In other embodiments, other types of data associated with the payer population 210 can be received. In some embodiments, payer demographic data 210a can include the age, sex, and/or known medical conditions of all members of the payer population. In some embodiments, payer demographic data 210a may manifest as an eligibility file with every member's age, sex, known conditions, as well as other demographic features. In some embodiments, historical claim data 210b may take the form of an anonymized file of most commercial medical claims in the United States, united under a common schema and sold by a vendor.

In some embodiments, a payer adjustment model 212 can be determined. In some embodiments, the payer adjustment model 212 can be based in part on a payer-specific coverage policy 212a and a payer-specific reinsurance policy 212b. In some embodiments, only a subset of the payer-specific coverage policy 212a and the payer-specific reinsurance policy 212b can be used to determine a payer adjustment model 212. In other embodiments, other types of customer-specific policy data can be used to determine the payer adjustment model 212. In some embodiments, the payer adjustment model 212 can consider that a payer may have a coverage policy that restricts the use of a novel therapy to a more limited set of clinical situations than another person. Such situations may result in a shift in the distribution of expected utilization and expected cost of a drug or therapy.

In some embodiments, a subscription prescription payment 214 for the therapy or pharmaceutical can be calculated based on the baseline disease model 208, the data associated with the payer population 210, and the payer adjustment model 212. In some embodiments, only a subset of the baseline disease model 208, the data associated with the payer population 210, and the payer adjustment model 212 can be used to calculate the subscription prescription payment 214. In other embodiments, additional considerations can be used to calculate the subscription prescription payment 214. In some embodiments, the subscription prescription payment 214 may be a per-member-per-month fee.

Although not depicted in FIG. 2, in some embodiments, the subscription prescription payment 214 can be configured to be displayed at a graphical user interface (GUI).

Although not depicted in FIG. 2, in some embodiments, the subscription prescription payment 214 can be updated periodically based on changes to the baseline disease model 208, the data associated with the payer population 210, and the payer adjustment model 212.

Although not depicted in FIG. 1, in some embodiments, the disease-specific demographic data 202, the metadata 204, the at least one model 206, the baseline disease model 208, the data associated with the payer population 210, and the payer adjustment model 212 may be weighted differently when calculating the subscription prescription payment 214. A user may manually define weights, or weights may be automatically defined using various machine learning algorithms.

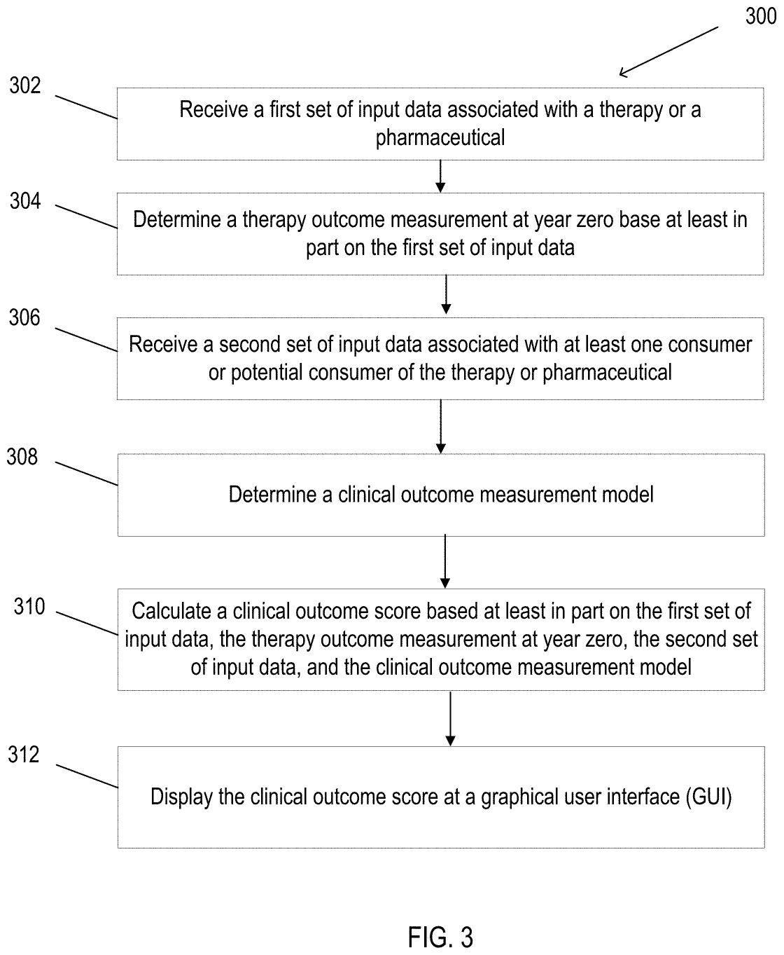

FIG. 3 illustrates a flow diagram of an exemplary methodology 300 for calculating a clinical outcome score in accordance with one example embodiment of the disclosure.

At block 302, a first set of input data associated with a therapy or a pharmaceutical can be received. The first set of input data can include any combination of therapy-specific data, for example, clinical trial data, scientific research data, patient advocacy data, and/or external testing data associated with the therapy or pharmaceutical.

At block 304, a therapy outcome measurement at year zero associated with the therapy or the pharmaceutical can be determined based at least in part on the first set of input data.

At block 306, a second set of input data associated with at least one consumer or potential consumer of the therapy or pharmaceutical can be received. The second set of input data can include any combination of historical medical claim data and/or medical charts of potential consumers.

At block 308, a clinical outcome measurement model associated with the therapy or pharmaceutical can be determined. In some embodiments, although not depicted in FIG. 3, the clinical outcome measurement model is configured to utilize a machine learning algorithm for calculating the clinical outcome score. In such embodiments, a first weight may be assigned to the first set of input data, a second weight may be assigned to the therapy outcome measurement at year zero, a third weight may be assigned to the second set of input data, and a fourth weight may be assigned to the clinical outcome measurement model.

At block 310, a clinical outcome score for the therapy or pharmaceutical can be calculated based at least in part on the first set of input data, the therapy outcome measurement at year zero, the second set of input data, and the clinical outcome measurement model. If a machine learning model is used to calculate the clinical outcome score, the clinical outcome score for the therapy or the pharmaceutical can be calculated based at least in part on the first set of input data, the therapy outcome measurement at year zero, the second set of input data, the clinical outcome measurement model, the first weight, the second weight, the third weight, and the fourth weight.

At optional block 312, the clinical outcome score for the therapy or pharmaceutical can be displayed at a graphic user interface (GUI).

Although not depicted in FIG. 3, the clinical outcome score may be transmitted over a network to a user device in real time, so as to provide updated information to a user.

Although not depicted in FIG. 3, the clinical outcome score may be used to train the machine learning algorithm so as to further increase the accuracy of the machine learning algorithm when it is used to calculate future subscription prescription payments.

An example that applies the methodology 300 provided in FIG. 3 is further provided. Many sources may provide data on the incidence of a disease within a population, for example, diagnosis information on medical claims, a number of sales of particular therapies for a disease, patient advocacy groups' anonymized or aggregated information on membership, and/or medical research on incidence in populations with different demographic characteristics. Given the demographics of a baseline population, a model for calculating a distribution for incidence of a disease in a total population can be constructed for a random subgroup of that population. That model could be augmented with treatment and diagnostic models to estimate a distribution of the number of people with the disease who may use a particular treatment. This augmented model may be known as the baseline disease model. For example, out of a random sample of 1,000 individuals from the overall U.S. population, the mean number of people who may seek a particular treatment for a certain disease may be 2. That random sampling can be supplemented with more specific information about a specific population to generate a user-specific estimate. Thus, if the 1,000 individuals were sampled from a particular population, the mean number of people who may seek a particular treatment for a certain disease may be 2.1. Then, based on the distribution of the estimated number of patients who may receive a particular treatment, a pricing system may be used to generate a subscription price that meets predetermined goals, such as reduced costs for payers and consumers, and increased revenues for treatment providers. The subscription price output may take the form of a price per member per month, or a price per member.

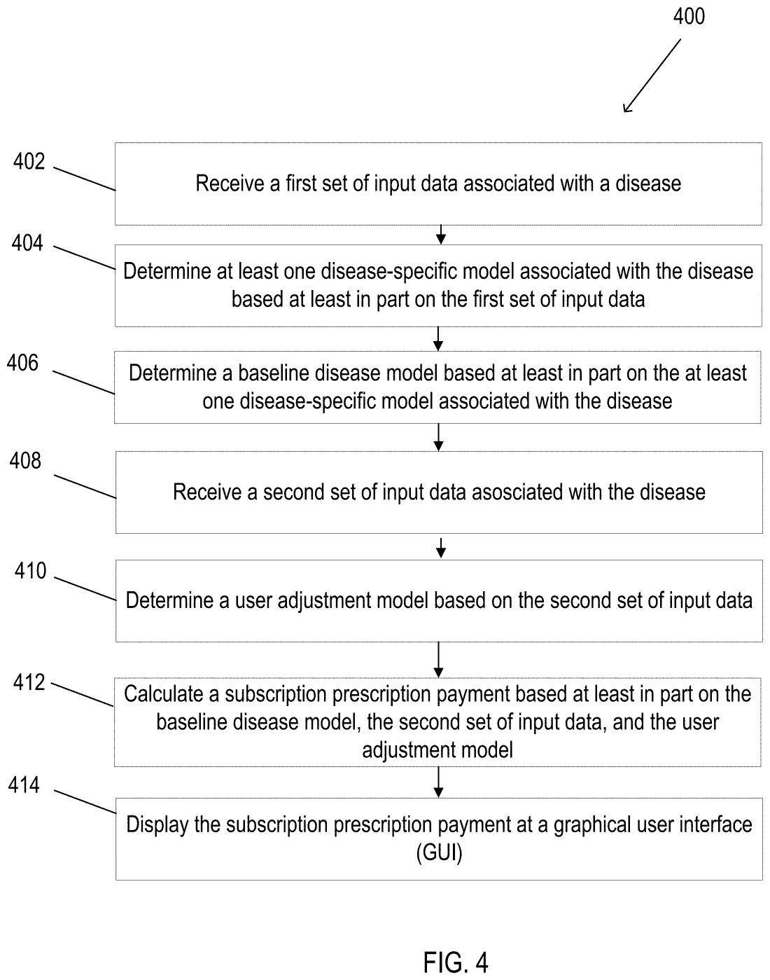

FIG. 4 illustrates a flow diagram of an exemplary methodology 400 for calculating a subscription prescription payment for a pharmaceutical or a therapy in accordance with one example embodiment of the disclosure.

At block 402, a first set of input data associated with a disease can be received. The first set of input data may include disease-specific demographic data and metadata associated with the disease. The demographic data can include any combination of claim data associated with the disease and a payer population, prescription data associated with the disease, patient advocacy data associated with the disease, and healthcare provider data associated with the disease. The metadata can include any combination of patient prevalence research associated with the disease, physician input associated with the disease, and generic screening and testing associated with the disease.

At block 404, at least one disease-specific model associated with the disease can be determined based at least in part on the first set of input data. The at least one disease-specific model can include any combination of a symptom progression model associated with the disease, a diagnostic pattern model associated with the disease, a treatment model associated with the disease, and a care management model associated with the disease.

At block 406, a baseline disease model can be determined based at least in part on the at least one disease-specific model associated with a disease.

At block 408, a second set of input data associated with the disease can be received. The second set of input data can include data associated with the payer population, such as payer demographic data and historical claim data associated with the payer population, and customer-specific policy data, such as payer-specific coverage policy data associated with the customer and payer-specific reinsurance policy data associated with the customer.

At block 410, a user adjustment model can be determined. The user adjustment model can be based on any combination of data associated with the second set of input data.

At block 412, a subscription prescription payment associated with a therapy or a pharmaceutical for treating a disease can be determined based at least in part on the baseline disease model, the second set of input data, and the user adjustment model. In some embodiments, although not depicted in FIG. 4, the subscription prescription payment may be calculated by utilizing a machine learning algorithm. In such embodiments, the first set of input data may be associated with a first weight, the plurality of disease-specific models may be associated with a second weight, the baseline disease model may be associated with a third weight, the second set of input data may be associated with a fourth weight, and the user adjustment model may be associated with a fifth weight. If a machine learning algorithm is used, the subscription prescription payment may be calculated based at least in part on the first set of input data, the plurality of disease-specific models, the baseline disease model, the second set of input data, the user adjustment model, the first weight, the second weight, the third weight, the fourth weight, and the fifth weight.

At optional block 414, the subscription prescription payment can be displayed at a graphical user interface (GUI).

Although not depicted in FIG. 4, the subscription prescription payment may be transmitted over a network to a user device in real time, so as to provide updated information to a user.

Although not depicted in FIG. 4, the subscription prescription payment may be used to train the machine learning algorithm so as to further increase the accuracy of the machine learning algorithm when it is used to calculate future subscription prescription payments.

An example that applies the methodology 400 provided in FIG. 4 is further provided. For example, a pharmaceutical treatment for a medical condition may be granted FDA approval on the basis of a clinical trial in which 90% of patients survived after 12 months. The baseline for FDA approval in similar circumstances may be 30%. This baseline may then serve as a clinical score and become a “prior” for treatment effectiveness at year zero. As new data emerges about the effectiveness of a treatment, the clinical score may be dynamically updated based on the new data. If the estimated survival rate associated with the treatment falls, the subscription price may correspondingly decrease. If the estimated survival rate associated with the treatment rises, the subscription price may correspondingly increase.

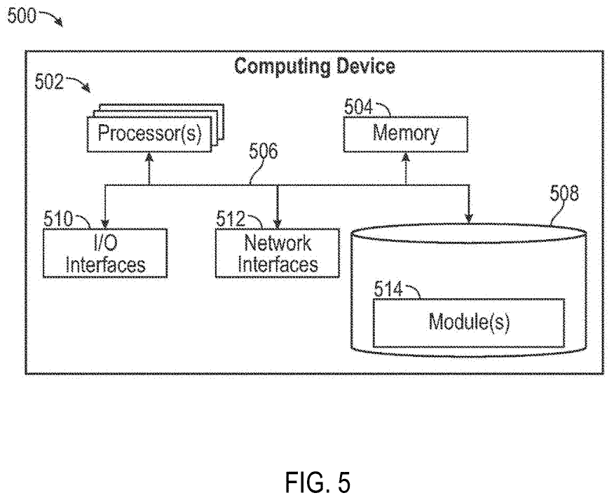

FIG. 5 illustrates an example computing device 500, in accordance with one or more embodiments of this disclosure. The computing device 500 may be representative of any devices described herein that may perform any active processing operations. The computing device 500 may include at least one processor 502 that executes instructions that are stored in one or more memory devices (referred to as memory 504). The instructions can be, for instance, instructions for implementing functionality described as being carried out by one or more modules and systems disclosed above or instructions for implementing one or more of the methods and processes disclosed above. The processor 502 can be embodied in, for example, a CPU, multiple CPUs, a GPU, multiple GPUs, a TPU, multiple TPUs, a multi-core processor, a combination thereof, and the like. In some embodiments, the processor(s) 502 can be arranged in a single processing device. In other embodiments, the processor(s) 502 can be distributed across two or more processing devices (for example, multiple CPUs; multiple GPUs; a combination thereof; or the like). A processor can be implemented as a combination of processing circuitry or computing processing units (such as CPUs, GPUs, or a combination of both). Therefore, for the sake of illustration, a processor can refer to a single-core processor; a single-processor with software multithread execution capability; a multi-core processor; a multi-core processor with software multithread execution capability; a multi-core processor with hardware multithread technology; a parallel processing (or computing) platform; and parallel computing platforms with distributed shared memory. Additionally, or as another example, a processor can refer to an integrated circuit (IC), an ASIC, a digital signal processor (DSP), an FPGA, a PLC, a complex programmable logic device (CPLD), a discrete gate or transistor logic, discrete hardware components, or any combination thereof designed or otherwise configured (for example, manufactured) to perform the functions described herein.

The processor(s) The processor(s) 502 can access the memory 504 by means of a communication architecture 506 (for example, a system bus). The communication architecture 506 may be suitable for the particular arrangement (localized or distributed) and type of the processor(s) 502. In some embodiments, the communication architecture 506 can include one or many bus architectures, such as a memory bus or a memory controller; a peripheral bus; an accelerated graphics port; a processor or local bus; a combination thereof, or the like. As an illustration, such architectures can include an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, a Peripheral Component Interconnect (PCI) bus, a PCI-Expressbus, a Personal Computer Memory Card International Association (PCMCIA) bus, a Universal Serial Bus (USB), and/or the like.

Memory components or memory devices disclosed herein can be embodied in either volatile memory or non-volatile memory or can include both volatile and non-volatile memory. In addition, the memory components or memory devices can be removable or non-removable, and/or internal or external to a computing device or component. Examples of various types of nontransitory storage media can include hard-disc drives, zip drives, CD-ROMs, digital versatile disks (DVDs) or other optical storage, magnetic cassettes, magnetic tape, magnetic disk storage or other magnetic storage devices, flash memory cards or other types of memory cards, cartridges, or any other non-transitory media suitable to retain the desired information and which can be accessed by a computing device.

As an illustration, non-volatile memory can include read-only memory (ROM), programmable ROM (PROM), electrically programmable ROM (EPROM), electrically erasable programmable ROM (EEPROM), or flash memory. Volatile memory can include random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). The disclosed memory devices or memories of the operational or computational environments described herein are intended to include one or more of these and/or any other suitable types of memory.

In addition to storing executable instructions, the memory 504 also can retain data, such as the clinical trial data 102, the scientific research data 104, the patient advocacy data 106, the external testing data 108, the therapy outcome measurement at year zero 110, the medical data 112, the clinical outcome measurement model 114, the medical review 116, the disease-specific demographic data 202, the metadata 204, the at least one model 206, the baseline disease model 208, the data associated with the payer population 210, and the payer adjustment model 212, among other data.

Each computing device 500 also can include mass storage 508 that is accessible by the processor(s) 502 by means of the communication architecture 506. The mass storage 508 can include machine-accessible instructions (for example, computer-readable instructions and/or computer-executable instructions). In some embodiments, the machine-accessible instructions may be encoded in the mass storage 508 and can be arranged in components that can be built (for example, linked and compiled) and retained in computer-executable form in the mass storage 508 or in one or more other machine-accessible non-transitory storage media included in the computing device 500. Such components can embody, or can constitute, one or many of the various modules 514 and systems disclosed herein. Execution of the modules and systems, individually or in combination, by at least one of the processor(s) 502, can cause the computing device 500 to perform any of the operations described herein.

Each computing device 500 also can include one or more input/output interface devices 510 (referred to as I/O interface 510) that can permit or otherwise facilitate external devices to communicate with the computing device 500. For instance, the I/O interface 510 may be used to receive and send data and/or instructions from and to an external computing device.

The computing device 500 also includes one or more network interface devices 512 (referred to as network interface(s) 512) that can permit or otherwise facilitate functionally coupling the computing device 500 with one or more external devices. Functionally coupling the computing device 500 to an external device can include establishing a wireline connection or a wireless connection between the computing device 500 and the external device. The network interface devices 512 can include one or many antennas and a communication processing device that can permit wireless communication between the computing device 500 and another external device. For example, between a vehicle and a smart infrastructure system, between two smart infrastructure systems, etc. Such a communication processing device can process data according to defined protocols of one or several radio technologies. The radio technologies can include, for example, 3G, Long Term Evolution (LTE), LTE-Advanced, 5G, IEEE 802.11, IEEE 802.16, Bluetooth, ZigBee, near-field communication (NFC), and the like. The communication processing device can also process data according to other protocols as well. The network interface(s) 512 may also be used to facilitate peer-to-peer ad-hoc network connections as described herein.

It should further be noted that the various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can include RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, include compact disc (CD), laser disc, optical disc, digital versatile disc (DVD), floppy disk, and Blu-ray disc (BD), where disks usually reproduce data optically with lasers. Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Certain aspects of the disclosure are described above with reference to block and flow diagrams of systems and methods according to various implementations. It will be understood that one or more blocks of the block diagrams and the flow diagrams, and combinations of blocks in the block diagrams and the flow diagrams, respectively, may be implemented by computer-executable program instructions. Likewise, some blocks of the block diagrams and the flow diagrams may not necessarily need to be performed in the order presented, or may not necessarily need to be performed at all, according to some implementations.

Computer-executable program instructions may be loaded onto a special-purpose computer or other particular machine, a processor, or other programmable data processing apparatus to product a particular machine, such that the instructions that execute on the computer, processor, or other programmable data processing apparatus create means for implementing one or more functions specified in the flow diagram block or blocks. These computer-executable program instructions may also be stored in a computer-readable storage media or memory that may direct a computer or other programmable data processing apparatus to function in a particular manner, such that the instructions stored in the computer-readable storage media produce an article of manufacture including instruction means that implement one or more functions specified in the flow diagram block or blocks. As an example, certain implementations may provide for a computer program product, comprising a computer-readable storage medium having a computer-readable program code or program instructions implemented therein, said computer-readable program code adapted to be executed to implement one or more functions specified in the flow diagram block or blocks. The computer-readable program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Though the disclosed examples include particular arrangements of a number of parts, components, features, and aspects, the disclosure is not limited to only those examples or arrangements shown. Any one or more of the parts, components, features, and aspects of the disclosure can be employed alone or in other arrangements of any two or more of the same.

Although certain product features, functions, components, and parts have been described herein in accordance with the teachings of the present disclosure, the scope of coverage is not limited thereto. On the contrary, this disclosure covers all embodiments of the teachings of the disclosure that fairly fall within the scope of permissible equivalents.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

Throughout this application, the term “include,” “include(s)” or “including” means “including but not limited to.” Note that certain embodiments may be described relating to a single component, but the corresponding description should be read to include embodiments of two or more components. Different features, variations, and multiple different embodiments are shown and described herein with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by this disclosure. It is indeed intended that the scope of this disclosure should be determined by a proper legal interpretation and construction of the disclosure, including equivalents, as understood by those of skill in the art relying upon the complete disclosure present at the time of filing.

Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or operations. Thus, such conditional language generally is not intended to imply that features, elements, and/or operations are in any way required for one or more implementations or that one or more implementations necessarily include logic for deciding, with or without user input or prompting, whether these features, elements, and/or operations are included or are to be performed in any particular implementation.

What has been described herein in the present specification and annexed drawings includes examples of systems and methods for determining a pricing model and calculating scores and payments associated therewith. It is, of course, not possible to describe every conceivable combination of components and/or methods for purposes of describing the various elements of the disclosure, but it can be recognized that many further combinations and permutations of the disclosed elements are possible. Accordingly, it may be apparent that various modifications can be made to the disclosure without departing from the scope thereof. In addition, or as an alternative, other embodiments of the disclosure may be apparent from consideration of the specification and annexed drawings, and practice of the disclosure as presented herein. It is intended that the examples put forth in the specification and annexed drawings be considered, in all respects, as illustrative and not limiting. Although specific terms are employed herein, they are used in a generic and descriptive sense only and not for purposes of limitation.

1. A computer-implemented method for determining a subscription prescription payment for a pharmaceutical or a therapy, comprising:
receiving a first set of input data associated with a disease;
assigning a first weight to the first set of input data;
determining a plurality of disease-specific models associated with the disease based at least in part on the first set of input data;
assigning a second weight to the plurality of disease-specific models;
determining a baseline disease model associated with the disease based on the plurality of disease-specific models;
assigning a third weight to the baseline disease model;
receiving a second set of input data;
assigning a fourth weight to the second set of input data;
determining a user adjustment model associated with a customer based at least in part on the second set of input data;
assigning a fifth weight to the user adjustment model; and
calculating the subscription prescription payment for the customer based at least in part on the baseline disease model, the second set of input data, the user adjustment model, the first weight, the second weight, the third weight, the fourth weight, and the fifth weight.

2. The computer-implemented method of claim 1, wherein the first set of input data comprises:
disease-specific demographic data; and
metadata associated with the disease.

3. The computer-implemented method of claim 2, wherein the disease-specific demographic data comprises:
claim data associated with the disease and a payer population;
prescription data associated with the disease;
patient advocacy data associated with the disease; and
healthcare provider data associated with the disease.

4. The computer-implemented method of claim 2, wherein the metadata associated with the disease comprises:
patient prevalence research associated with the disease;
physician input associated with the disease; and
generic screening and testing associated with the disease.

5. The computer-implemented method of claim 1, wherein the plurality of disease-specific models comprises:
a symptom progression model associated with the disease;
a diagnostic pattern model associated with the disease;
a treatment model associated with the disease; and
a care management model associated with the disease.

6. The computer-implemented method of claim 1, wherein the second set of input data comprises:
data associated with a payer population; and
customer-specific policy data.

7. The computer-implemented method of claim 6, wherein the data associated with the payer population comprises:
payer demographic data associated with the payer population; and
historical claim data associated with the payer population.

8. The computer-implemented method of claim 6, wherein the customer-specific policy data comprises:
payer-specific coverage policy data associated with the customer; and
payer-specific reinsurance policy data associated with the customer.

9. A system for determining a subscription prescription payment for a pharmaceutical or a therapy comprising, a computing device comprising storage and processing circuitry configured to:
receive a first set of input data associated with a disease;
determine a plurality of disease-specific models associated with the disease based at least in part on the first set of input data;
determine a baseline disease model associated with the disease based on the plurality of disease-specific models;
receive a second set of input data;
determine a user adjustment model associated with a customer based at least in part on the second set of input data; and
calculate the subscription prescription payment for the customer based at least in part on the baseline disease model, the second set of input data, and the user adjustment model.

10. The system of claim 9, wherein the first set of input data comprises:
disease-specific demographic data comprising:
claim data associated with the disease and a payer population;
prescription data associated with the disease;
patient advocacy data associated with the disease; and
healthcare provider data associated with the disease; and
metadata associated with the disease comprising:
patient prevalence research associated with the disease;
physician input associated with the disease; and
generic screening and testing available with the disease.

11. The system of claim 9, wherein the plurality of disease-specific models comprises:
a symptom progression model associated with the disease;
a diagnostic pattern model associated with the disease;
a treatment model associated with the disease; and
a care management model associated with the disease.

12. The system of claim 9, wherein the second set of input data comprises:
data associated with a payer population comprising:
payer demographic data associated with the payer population; and
historical claim data associated with the payer population; and
customer-specific policy data comprising:
payer-specific coverage policy data associated with the customer; and
payer-specific reinsurance policy data associated with the customer.

13. The system of claim 9, wherein the subscription prescription payment is calculated by using a machine learning algorithm.

14. The system of claim 9, wherein the first set of input data is associated with a first weight, the plurality of disease-specific models is associated with a second weight, the baseline disease model is associated with a third weight, the second set of input data is associated with a fourth weight, and the user adjustment model may be associated with a fifth weight, and wherein the subscription prescription payment is calculated based at least in part on the first set of input data, the plurality of disease-specific models, the baseline disease model, the second set of input data, the user adjustment model, the first weight, the second weight, the third weight, the fourth weight, and the fifth weight.

15. A computer-implemented method for determining a clinical outcome score associated with a therapy or a pharmaceutical, the method comprising:
receiving a first set of input data associated with the therapy or the pharmaceutical;
determining a therapy outcome measurement at year zero based at least in part on the first set of input data;
receiving a second set of input data associated with consumers or potential consumers;
determining a clinical outcome measurement model associated with the therapy or the pharmaceutical; and
calculating the clinical outcome score associated with the therapy or the pharmaceutical based at least in part on the first set of input data, the therapy outcome measurement at year zero, the second set of input data, and the clinical outcome measurement model.

16. The computer-implemented method of claim 15, wherein the first set of input data comprises therapy-specific data associated with the therapy or the pharmaceutical, and wherein the therapy-specific data comprises:
clinical trial data;
scientific research data;
patient advocacy data; and
external testing data.

17. The computer-implemented method of claim 15, wherein the second set of input data comprises historical claim data and medical charts of potential consumers of the therapy or the pharmaceutical.

18. The computer-implemented method of claim 15, wherein the clinical outcome measurement model is configured to utilize a machine learning algorithm for calculating the clinical outcome score.

19. The computer-implemented method of claim 18, wherein a first weight is associated with the first set of input data, a second weight is associated with the therapy outcome measurement at year zero, a third weight is associated with the second set of input data, and a fourth weight is associated with the clinical outcome measurement model.

20. The computer-implemented method of claim 19, wherein the clinical outcome score is calculated based at least in part on the first set of input data, the therapy outcome measurement at year zero, the second set of input data, the clinical outcome measurement model, the first weight, the second weight, the third weight, and the fourth weight.
